(19) United States
(12) Patent Application Publication
(10) Pub. No.: US 2019/0060610 A1
(43) Pub. Date: Feb. 28, 2019

(54) FOLEY CATHETER

(71) Applicant: MedAll Biotech Co., Ltd., Taipei (TW)

(72) Inventor: Chi-Hsien SHE, Taipei (TW)

(21) Appl. No.: 15/787,263

(22) Filed: Oct. 18, 2017

(30) Foreign Application Priority Data
Aug. 24, 2017 (TW) 106212508

(51) Int. Cl. A61M 25/00; A61M 25/10; A61M 3/02; A61M 25/01

(52) U.S. Cl. CPC A61M 25/0017; A61M 25/10184; A61M 2025/0188; A61M 25/0043; A61M 25/01; A61M 3/0283

(57) ABSTRACT

Disclosed is a Foley catheter which comprises a main tube, a positioning balloon portion, a urine drainage branch tube, an irrigation branch tube and a breaking portion. The main tube has a urine drainage lumen and an irrigation lumen. One end of the main tube has the positioning balloon portion communicated with the irrigation lumen, and the other end extends to form the urine drainage branch tube communicated with the urine drainage lumen and the irrigation branch tube communicated with the irrigation lumen. The breaking portion is formed at any one of positions including the position at the main tube, the urine drainage branch tube and the middle section of the irrigation branch tube, wherein the breaking portion is disposed in such a manner that the breaking portion will be broken when a tweak force exceeding a predetermined value is applied so that the Foley catheter is divided into two sections or the fluid in the positioning balloon portion flows out.

### FIELD OF THE INVENTION

The present invention relates to a Foley catheter, and more particularly to a Foley catheter which can break by itself as receiving a tweaking accidentally.

### BACKGROUND OF THE INVENTION

Foley catheter is an artificial catheter that helps draining urine from the bladder. Foley catheter is a tube usually made of rubber, silicone or plastic, and is placed in the bladder and connected to the outside of the body through the urethra for urine drainage. Foley catheters are placed in approximately millions of patients per year through catheterizations including temporary catheterization and long-term catheterization. Temporary catheterization is applied to the prevention of postoperative wound-healing disturbance and urine output monitoring, while long-term catheterization is applied to the prevention of urinary incontinence and urinary retention. Procedure for Foley catheter placement includes inserting the front-end of Foley catheter into the bladder through the urethra and connecting a urinary drainage bag to the end of the Foley catheter to allow urine to drain into the urinary drainage bag. Moreover, for preventing accidental removal of Foley catheter, balloon disposed in the introducing end of the Foley catheter is inflated with fluid so as to keep the Foley catheter securely stuck in the bladder.

However, when the Foley catheter receives a tweaking, an inflated balloon in the introducing end thereof stuck in the bladder may be squeezed into the urethra so as to induce injury to urethral mucosa. The injury of urethral mucosa not only increases the urethra infection probability but also prolongs the length of stay in hospital, thereby increasing medical costs and expenses.

### SUMMARY OF THE INVENTION

As mentioned above, since the Foley catheter in the prior arts may cause damage and infection to the human body as receiving a tweaking accidentally, it is necessary to improve the conventional Foley catheter.

Thus, an objective of the present invention is to provide a Foley catheter which can prevent injuries due to the accidental tweaking of the Foley catheter.

The technical means adopted by the present invention to overcome the drawbacks in the prior art is to provide a Foley catheter, comprising a main tube whose two ends are respectively an introducing end and a drainage end, wherein a urine drainage lumen and an irrigation lumen are provided within a tube body of the main tube in such a manner that the urine drainage lumen and the irrigation lumen are disposed between the introducing end and the drainage end, a positioning balloon portion disposed in the introducing end and being communicated with the irrigation lumen, a urine drainage branch tube communicating with and extending from the drainage end, the urine drainage branch tube being communicated with the urine drainage lumen, an irrigation branch tube communicating with and extending from the drainage end, the irrigation branch tube being communicated with the irrigation lumen, and a breaking portion formed at any one of positions including the position between the introducing end and the drainage end, the position between two ends of the urine drainage branch tube, and the position between two ends of the irrigation branch tube, wherein the breaking portion is disposed in such a manner that the breaking portion will be broken when a tweak force exceeding a predetermined value is applied.

In one embodiment of the present invention, the breaking portion is a tube with a circular notch formed circularly on a tube wall.

In one embodiment of the present invention, the breaking portion is a tube made of fragile material.

In one embodiment of the present invention, the shortest distance of the tube wall at the circular notch of the breaking portion is 0.13˜0.16 cm.

In one embodiment of the present invention, further including an anti-pulling part detachably disposed at the breaking portion or in surrounds of the breaking portion, and the anti-pulling portion is arranged to share a tweak force applied to the breaking portion as the Foley catheter receives a tweaking.

In one embodiment of the present invention, further including a suction unit communicated with the positioning balloon, wherein the suction unit is arranged to draw fluid from the positioning balloon portion as a predetermined breaking force is applied to the breaking portion.

In one embodiment of the present invention, the main tube is a tube with a right lumen and a left lumen, and the length of the urine drainage branch tube is greater than the length of the injecting branch tube, and the breaking portion is formed at the tube wall of the urine drainage branch tube.

In one embodiment of the present invention, the main tube is a tube with an inner lumen and an outer lumen, and the irrigation lumen is disposed at the outer border of the urine drainage lumen, and the breaking portion is formed at the tube wall of the irrigation lumen of the main tube.

By means of the technology of the present invention, the breaking portion is provided such that the positioning balloon portion and the portion connected to the urinary drainage bag can be immediately separately from each other when the Foley catheter receives a tweaking accidentally so as to ensure the retention of the positioning balloon portion in the bladder thereby preventing the damage to the urethra. Alternatively, the Foley catheter can break by the fracture of the irrigation lumen or the tube wall of the irrigation branch tube in such a manner that the positioning balloon portion shrinks due to the leakage of the fluid in the positioning balloon portion, thereby greatly reducing the probability of the damage caused by the leakage fluid passing through the urethra.

### BRIEF DESCRIPTION OF THE DRAWINGS

FIG. 1 is an isometric view illustrating a Foley catheter according to one embodiment of the present invention

FIG. 2 is a sectional schematic view illustrating a Foley catheter in which fluid is drained into the positioning balloon portion according to the embodiment of the present invention.

FIGS. 3a and 3b is a schematic view illustrating an application of the Foley catheter according to the embodiment of the present invention.

FIG. 4 is a sectional schematic view illustrating a Foley catheter in which fluid is drained into the positioning balloon portion according to the other embodiment of the present invention.

FIGS. 5a-5c is a schematic view illustrating an application of the Foley catheter according to the other embodiment of the present invention.

FIG. 6 is a schematic view illustrating an application of the Foley catheter according to the other embodiment of the present invention.

### DETAILED DESCRIPTION OF THE PREFERRED EMBODIMENTS

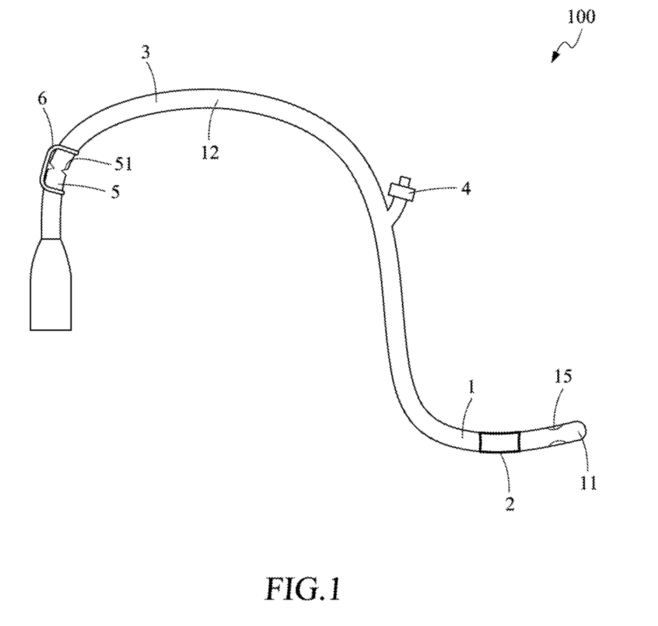

The preferred embodiments of the present invention are described below with reference to FIGS. 1-6. The description is only the explanation of the preferred embodiments, and is not the limitation of the implementation of the present invention.

As shown in FIG. 1, according to the first embodiment of the present invention, a Foley catheter 100 comprises a main tube 1, a positioning balloon portion 2, a urine drainage branch tube 3, an irrigation branch tube 4 and a breaking portion 5.

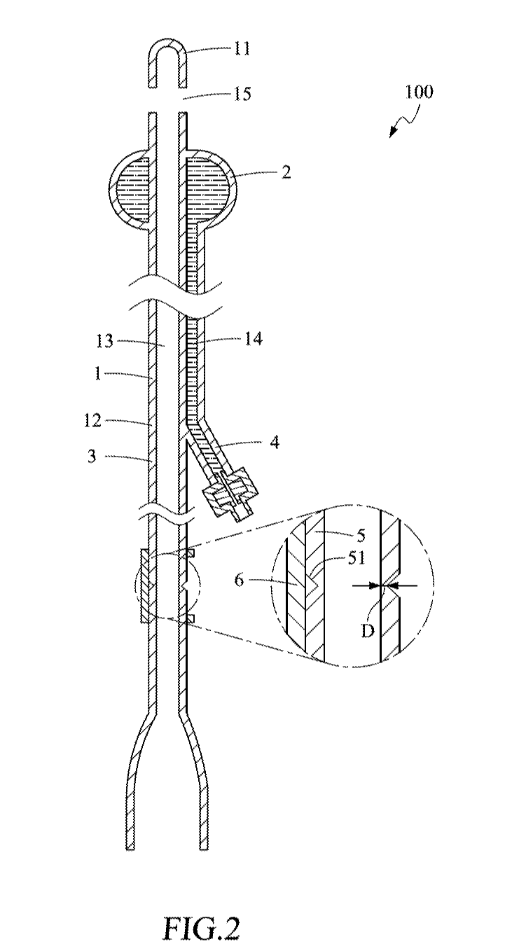

As shown in FIGS. 1 and 2, the main tube 1 is a tube body having a size smaller than the size of the urethra of the predetermined human body in order to be introducible into the human body via the urethra. Two ends of the main tube 1 are an introducing end 11 and a drainage end 12. In one embodiment of the present invention, the main tube 1 is a double lumen tube, wherein a urine drainage lumen 13 and an irrigation lumen 14 are provided within a tube body of the main tube 1 in such a manner that the urine drainage lumen 13 and the irrigation lumen 14 are disposed between the introducing end 11 and the drainage end 12. However, the present invention is not limited to this. The main tube 1 can be a triple lumen tube in which an additional drainage lumen is disposed to inject the cleaning fluid to the bladder. In the present embodiment, the tip of the introducing end 11 is provided with two urine drainage holes 15 for draining urine in the bladder into the urine drainage lumen 13.

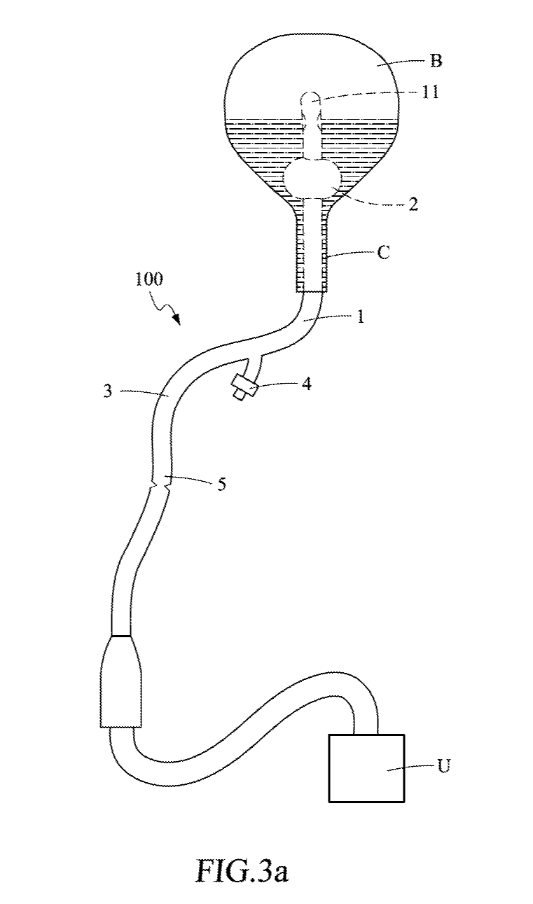

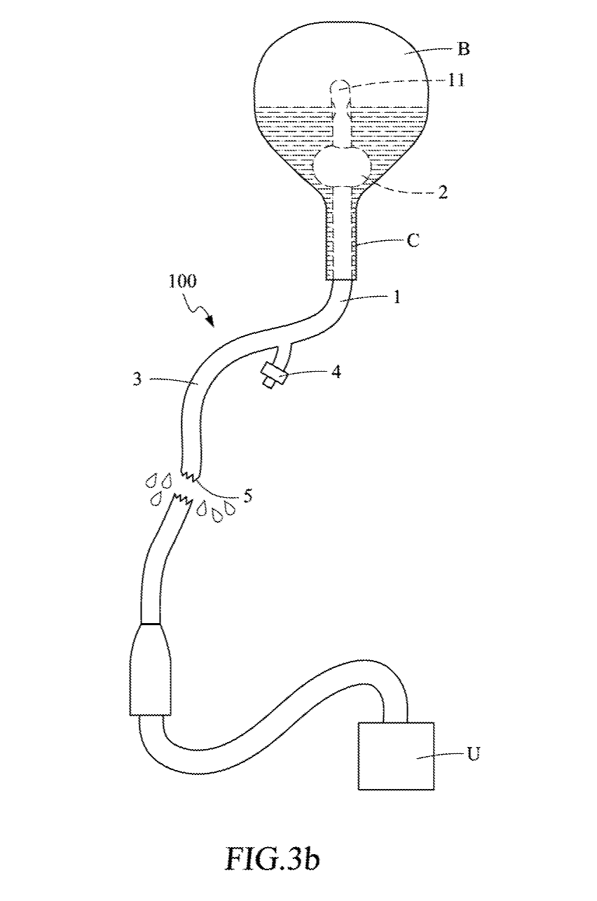

As shown in FIGS. 1 and 2, the positioning balloon portion 2 is disposed at the introducing end 11 and the rear side of the urine drainage holes 15. The positioning balloon portion 2 is a balloon formed by a film, and the lumen of the balloon is communicated with the irrigation lumen 14. When not in use, the positioning balloon portion 2 is attached to the surrounds of the main tube 1 so that the positioning balloon portion 2 and the main tube 1 together can pass through the urethra C to a bladder B with a size smaller than the urethra (as shown in FIG. 1). After the introducing end 11 and the positioning balloon portion 2 insert into the bladder, the positioning balloon portion 2 inflates to a size greater than the size of the urethra by the fluid through the irrigation lumen 14 to keep the introducing end 11 stuck in the bladder B (as shown in FIGS. 2-3B). However, the present invention is not limited to this. The positioning balloon portion 2 can also be inflated with gas.

As shown in FIGS. 1 and 2, the urine drainage branch tube 3 and the irrigation branch tube 4 respectively extends to connect to the drainage end 12. Moreover, the urine drainage branch tube 3 communicates with the urine drainage lumen 13, and the rear end of the urine drainage branch tube 3 is provided with a connecter connecting to the urinary drainage bag U for draining urine in the urine drainage lumen 13 into the urinary drainage bag U. The irrigation branch tube 4 communicates with the irrigation lumen 14. In the present embodiment, the main tube 1, the urine drainage branch tube 3, the irrigation branch tube 4 and the breaking portion 5 can be flexible silicone tubes integrally formed. However, the present invention is not limited to this. The main tube 1, the urine drainage branch tube 3, the irrigation branch tube 4 and the breaking portion 5 can further be limited to silicone tubes or plastic tubes which are made from antimicrobial materials.

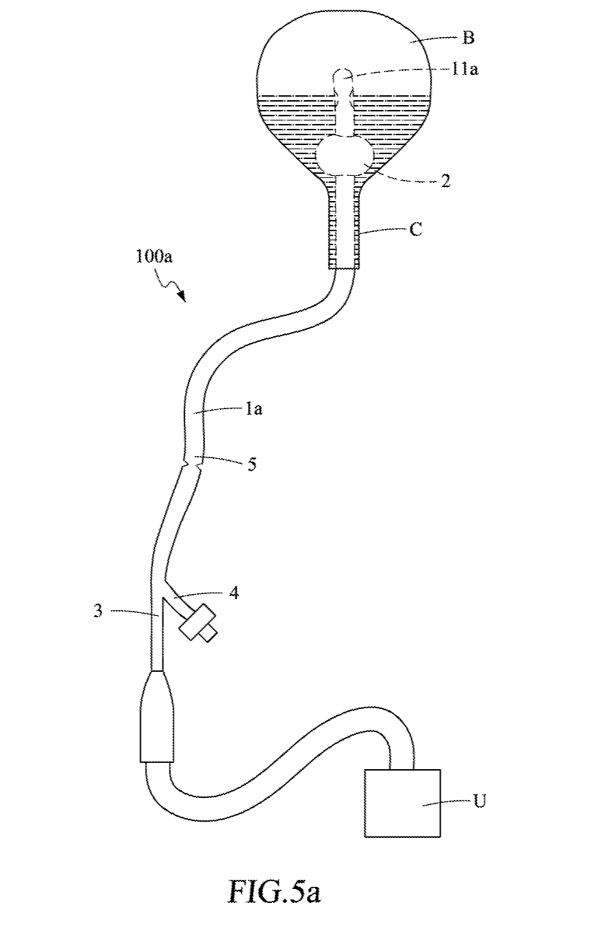

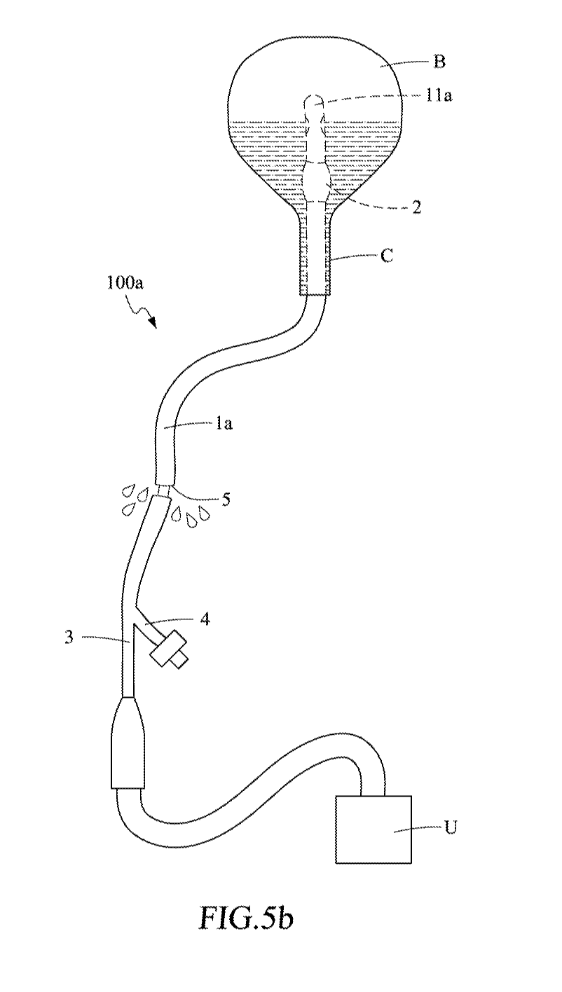

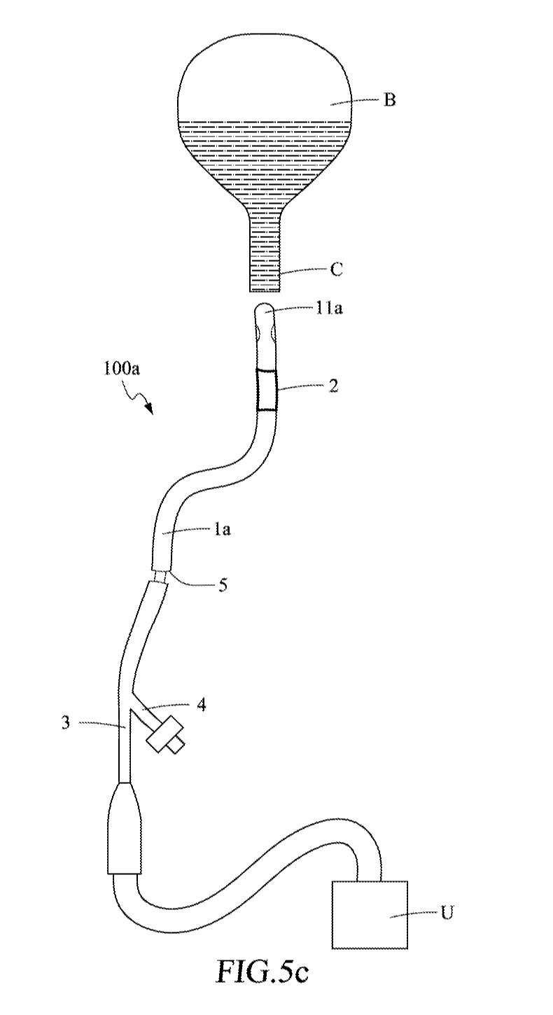

As shown in FIGS. 1-5C, the breaking portion 5 is formed at any one of the positions including the position between the introducing end 11 and the drainage end 12, the position between two ends of the urine drainage branch tube 3, and the position between two ends of the irrigation branch tube 4. The breaking portion 5 is disposed in such a manner that the breaking portion 5 will be broken when a tweak force exceeding a predetermined value is applied so that the Foley catheters 100 is divided into two sections (as shown in FIG. 3B), while the front section of the Foley catheters 100 remains in the bladder for urine drainage. Furthermore the fluid in the positioning balloon portion 2 is drained out through the irrigation lumen 14 as the tube wall of the irrigation lumen 14 or the irrigation branch tube 4 breaks, and thereby the positioning balloon portion 2 shrinks to a size less than the size of the urethra due to the output of the fluid (as shown in FIG. 5C).

Selectively, as shown in FIGS. 1 to 5C, the breaking portion 5 is a tube with a circular notch 51 formed circularly on a tube wall, and will be broken when a tweak force exceeding a predetermined value is applied. Experiments confirmed that the shortest distance D of the tube wall at the circular notch of the breaking portion 5 can be 0.13˜0.16 cm (as shown in FIG. 2). In the present embodiment, as shown in FIG. 2, the shortest distance D of the tube wall at the circular notch of the breaking portion 5 is 0.15 cm. However, the present invention is not limited to this. The breaking portion 5 can be formed in other ways, e.g. the breaking portion 5 being a small section of a tube (not shown) made of fragile material, and it will be broken when a tweak force exceeding a predetermined value is applied.

As shown in FIGS. 1-3B, the main tube 1 is a tube with a right lumen and a left lumen which are respectively the urine drainage lumen 13 and the irrigation lumen 14. The urine drainage branch tube 3 is arranged to be fixed in a predetermined site of the human body. The length of the urine drainage branch tube 3 is greater than the length of the injecting branch tube 4. The breaking portion 5 is formed at the rear tube wall of the urine drainage branch tube 3 fixed in a predetermined site of the human body. FIG. 3A illustrates the Foley catheters 100 being fixed in the human body by fixing the positioning balloon portion 2 in the bladder B and fixing the urine drainage branch tube 3 in the human body. FIG. 3B illustrates the Foley catheters 100 broken at the breaking portion 5 after received a tweak force making the urine drainage branch tube split into two sections, one of which is fixed in the human body including the main tube 1, the positioning balloon portion 2 and the portion of the urine drainage branch tube 3 preceding the breaking portion 5, and the other separated from the human body including the portion of the urine drainage branch tube 3 following the breaking portion 5 and the urinary drainage bag U connected to the urine drainage branch tube 3. By means of the structure mentioned above, the positioning balloon portion 2 can be prevented from being squeezed into the urethra, because the introducing end 11 of the main tube 1 can still be stuck in the bladder B by the positioning balloon portion 2 when the Foley catheters 100 breaks, and the urine drainage holes 15 can still drain the urine to the broken site of the breaking portion 5 of the urine drainage branch tube 3 resulting in urine-leaking.

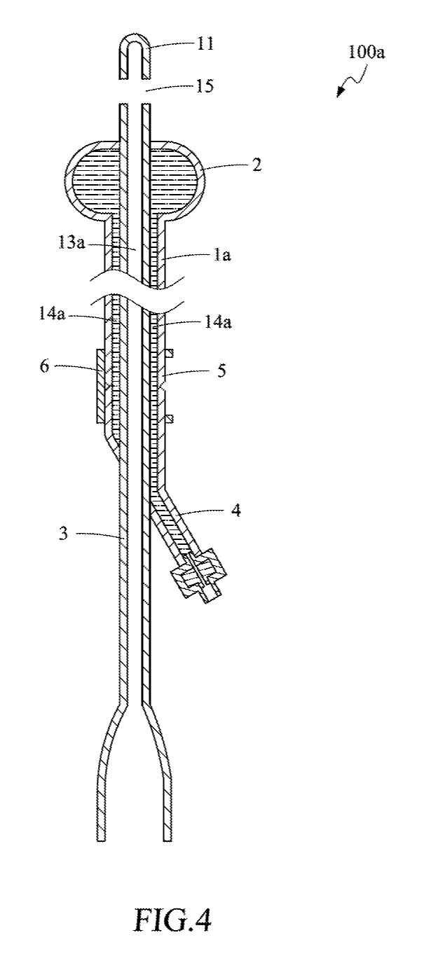

FIGS. 4-5C illustrate a Foley catheter 100a according to the other embodiment of the present invention, in which the main tube 1a is a tube with an inner lumen and an outer lumen. The irrigation lumen 14a disposed at the outside of the urine drainage lumen 13 is the outer lumen. The main tube 1a is arranged to be fixed in a predetermined site in the human body. The breaking portion 5 is a tube with a circular notch 51 formed circularly on a tube wall of the irrigation lumen 14a of the main tube 1a. As shown in FIG. 5A, when the Foley catheter 100a is fixed in the human body, the Foley catheter 100a breaks from the breaking portion 5 as a breaking force is applied in such a manner that the tube wall of the irrigation lumen 14a is divided into two sections, as shown in FIG. 5B. The fluid in the positioning balloon portion 2 is drained through the irrigation lumen 14a to the broken site of the breaking portion 5 of the main tube 1a so that the positioning balloon portion 2 shrinks. As shown in FIG. 5C, the positioning balloon portion 2 of the Foley catheter 100a shrunk to the original size which is smaller than the size of the urethra C can pass through the urethra and thus can draw out of the body together with the introducing end 11a of the Foley catheter 100a.

All of the above are examples. However, the present invention is not limited to this. The breaking portion 5 can be disposed at various Foley catheters in other ways, and also can be disposed at various site on the Foley catheters 100, 100a in multiple groups for achieving multiple protection from injuries caused by tweaked Foley catheters 100, 100a.

Preferably, as shown in FIGS. 1, 2-4, the Foley catheters 100 and 100a further include an anti-pulling part 6 which is detachably disposed at the breaking portion 5 or in surrounds of the breaking portion 5. The anti-pulling portion 6 is arranged to share a tweak force applied to the breaking portion 5 as the Foley catheter 100 receives a tweaking for preventing the breaking portion 5 from being broken by non-intentional tweaking.

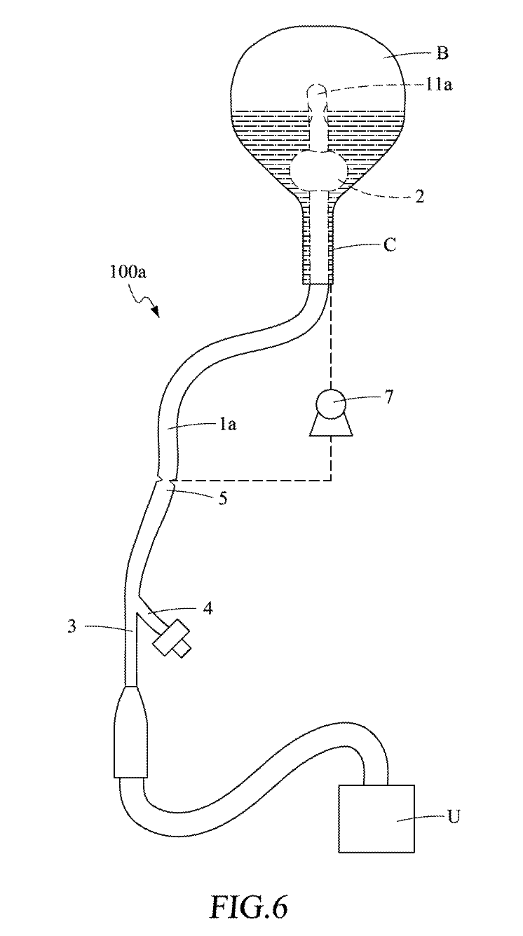

Preferably, as shown in FIG. 6, the Foley catheter 100a further includes a suction unit 7 communicated with the positioning balloon 2 directly, or communicated with the positioning balloon 2 via the main tube 1a or the irrigation branch tube 4. The suction unit is arranged to draw fluid from the positioning balloon portion as a predetermined breaking force is applied to the breaking portion to help the fluid in the positioning balloon 2 to flow out faster.

The above description is only the explanation of the preferred embodiments of the present invention. However, a person with ordinary skill in the art may make various modifications to the present invention. Those modifications shall still fall within the spirit and the scope defined by the appended claims

What is claimed is:

1. A Foley catheter, comprising:
a main tube whose two ends are respectively an introducing end and a drainage end, wherein a urine drainage lumen and an irrigation lumen are provided within a tube body of the main tube in such a manner that the urine drainage lumen and the irrigation lumen are disposed between the introducing end and the drainage end,
a positioning balloon portion disposed in the introducing end and being communicated with the irrigation lumen,
a urine drainage branch tube communicating with and extending from the drainage end, the urine drainage branch tube being communicated with the urine drainage lumen,
an irrigation branch tube communicating with and extending from the drainage end, the irrigation branch tube being communicated with the irrigation lumen, and
a breaking portion formed at any one of positions including the position between the introducing end and the drainage end, the position between two ends of the urine drainage branch tube, and the position between two ends of the irrigation branch tube, wherein the breaking portion is disposed in such a manner that the breaking portion will be broken when a tweak force exceeding a predetermined value is applied.

2. The Foley catheter of claim 1, wherein the breaking portion is a tube with a circular notch formed circularly on a tube wall.

3. The Foley catheter of claim 1, wherein the breaking portion is a tube made of fragile material.

4. The Foley catheter of claim 2, wherein the shortest distance of the tube wall at the circular notch of the breaking portion is 0.13˜0.16 cm.

5. The Foley catheter of claim 1, further including an anti-pulling part detachably disposed at the breaking portion or in surrounds of the breaking portion, and the anti-pulling portion is arranged to share a tweak force applied to the breaking portion as the Foley catheter receives a tweaking.

6. The Foley catheter of claim 1, further including a suction unit communicated with the positioning balloon, wherein the suction unit is arranged to draw fluid from the positioning balloon portion as a predetermined breaking force is applied to the breaking portion.

7. The Foley catheter of claim 1, wherein the main tube is a tube with a right lumen and a left lumen, and the length of the urine drainage branch tube is greater than the length of the injecting branch tube, and the breaking portion is formed at the tube wall of the urine drainage branch tube.

8. The Foley catheter of claim 1, wherein the main tube is a tube with an inner lumen and an outer lumen, and the irrigation lumen is disposed at the outer border of the urine drainage lumen, and the breaking portion is formed at the tube wall of the irrigation lumen of the main tube.
